(19) United States
(12) Patent Application Publication
(10) Pub. No.: US 2006/0270677 A1
(43) Pub. Date: Nov. 30, 2006

(54) Method for the treatment of noise phobia in companion animals

(75) Inventors: Annmarie Enos, Cranbury, NJ (US); Cecil Mark Eppler, Langhorne, PA (US); Dennis William Powell, Courtlandt Manor, NY (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/440,779

(22) Filed: May 25, 2006

Related U.S. Application Data
(60) Provisional application No. 60/684,669, filed on May 26, 2005.

(51) Int. Cl. A61K 31/519; A61K 31/503

(52) U.S. Cl. 514/248; 514/259.3

(57) ABSTRACT

The present invention provides a method for the non-sedative treatment of noise phobia in a companion animal, particularly a dog, which comprises providing said animal with a therapeutically effective amount of N-methyl-N-[3-(3-methyl-1,2,4-triazolo-[4,3-b]pyridazin-6-yl)phenyl]acetamide or 7-(3-pyridyl)pyrazolo[1,5-a]pyrimidine-3-carbonitrile.

This application claims the benefit under 35 U.S.C. §119(e) to co-pending U.S. provisional application No. 60/684669, filed May 26, 2005, which is hereby incorporated by reference in its entirety.

### BACKGROUND OF THE INVENTION

Noise and thunderstorm phobias are among the most commonly recognized disorders associated with panic or phobic responses in companion animals such as dogs, cats or horses, particularly dogs. Thunderstorms, fireworks, gunfire, car backfire, etc. frequently induce undesirable nonspecific clinical symptons in companion animals, particularly dogs, such as salivating, defecating, urinating, destroying, escaping, hiding trembling, vocalizing and the like. Known treatments for general anxiety behavior in companion animals generally involve either a long period of onset, i.e. 3-4 weeks, or if quick-acting, cause sedation and/or ataxia. However, most companion animal owners and veterinarians would prefer to treat their animals suffering from noise phobia with a method which does not promote sedation or ataxia and which is effective within an hour or two of administration.

Therefore, it is an object of this invention to provide a therapeutically effective method for the treatment of noise phobia in a companion animal which is non-sedative.

It is also an object of the invention to provide a veterinary composition which is useful for the treatment of noise phobia in a companion animal.

It is a feature of the invention that the method for the effective treatment of noise phobia in a companion animal, particularly a dog, is quick acting and does not cause ataxia.

Other objects and features of the invention will be come more apparent from the detailed description set forth hereinbelow.

### SUMMARY OF THE INVENTION

The present invention provides a method for the treatment and prevention of noise phobia in a companion animal which comprises providing said animal with a therapeutically effective amount of N-methyl-N-[3-(3-methyl-1,2,4-triazolo-[4,3-b]pyridazin-6-yl)phenyl]acetamide or 7-(3-pyridyl)pyrazolo[1,5-a]pyrimidine-3-carbonitrile.

Also provided is a veterinary composition for the effective treatment of noise phobia in a companion animal.

### DETAILED DESCRIPTION OF THE INVENTION

Owners of companion animals and veterinarians strive to find means to control noise phobia in their animals such as dogs, cats and horses, particularly dogs. Noise phobia behaviors may include hiding, scanning, urinating, defecating, panting, chewing, pacing, escaping, trembling, vocalizing and the like. Known therapies used for noise phobia incude off-label therapies such as clomipramine, amitriptyline and buspirone which can take more than 3-4 weeks before an effect is apparent, or the use of benzodiazepines, acepromazine or antidepressants which act more quickly but often cause sedation and ataxia.

Surprisingly, it has now been found that N-methyl-N-[3-(3-methyl-1,2,4-triazolo-[4,3-b]pyridazin-6-yl)phenyl]acetamide (acetamide) or 7-(3-pyridyl)pyrazolo[1,5-a]pyrimidine-3-carbonitrile (carbonitrile) is useful for the therapeutic treatment and prevention of noise phobia in a companion animal without causing sedation or ataxia and with a shortened period of onset. Accordingly the present invention provides a method for the treatment and prevention of noise phobia in a companion animal which comprises providing said animal with a therapeutically effective amount of N-methyl-N-[3-(3-methyl-1,2,4-triazolo-[4,3-b]pyridazin-6-yl)phenyl]acetamide or 7-(3-pyridyl)pyrazolo[1,5-a]pyrimidine-3-carbonitrile. Advantageously, the method of the invention is effective within 1-2 hours and is non-sedating and non-debilitating.

As used in the spefication and claims the term “acetamide” designates N-methyl-N-[3-(3-methyl-1,2,4-triazolo-[4,3-b]pyridazin-6-yl)phenyl]acetamide and the term “carbonitrile” designates 7-(3-pyridyl)pyrazolo[1,5-a]pyrimidine-3-carbonitrile.

The term “providing” as used herein with respect to providing a compound or substance embraced by the invention, designates either directly administering such a compound or substance, or administering a prodrug, derivative or analog which forms an equivalent amount of the compound or substance within the body.

The therapeutically effective amount provided in the treatment of noise phobia may vary according to the specific condition(s) being treated, the size, age and response pattern of the companion animal, the severity of the disorder, the judgment of the attending veterinarian or the like. In general, effective amounts for daily oral administration may be about 0.01 to 1,000 mg/kg, preferably about 0.5 to 500 mg/kg and effective amounts for parenteral administration may be about 0.1 to 100 mg/kg, preferably about 0.5 to 50 mg/kg.

Companion animals suitable for use in the method of invention include dogs, cats, horses, hamsters, guinea pigs, or any common domesticated pet, preferably dogs.

The compound N-methyl-N-[3-(3-methyl-1,2,4-triazolo-[4,3-b]pyridazin-6-yl)phenyl]acetamide and a method to prepare said compound is described in U.S. Pat. No. 4,767,765. The compound 7-(3-pyridyl)pyrazolo[1,5-a]pyrimidine-3-carbonitrile and a method to prepare said compound is described in U.S. Pat. No. 4,2281,000.

In actual practice, the compounds are provided by administering the compound or a precursor thereof in a solid or liquid form, either neat or in combination with one or more conventional veterinary pharmaceutical carriers or excipients. Accordingly, the present invention provides a veterinary composition which comprises a veterinary pharmaceutically acceptable carrier and an effective amount of N-methyl-N-[3-(3-methyl-1,2,4-triazolo-[4,3-b]pyridazin-6-yl)phenyl]acetamide or 7-(3-pyridyl)pyrazolo[1,5-a]pyrimidine-3-carbonitrile.

Solid carriers suitable for use in the composition of the invention include one or more substances which may also act as flavoring agents, lubricants, solubilizers, suspending agents, fillers, glidants, compression aides, binders, tablet-disintegrating agents or encapsulating materials. In powders, the carrier may be a finely divided solid which is in admixture with a finely divided acetamide or carbonitrile active ingredient. In tablets, the acetamide or carbonitrile compound may be mixed with a carrier having the necessary compression properties in suitable proportions and compacted in the shape and size desired. Said powders and tablets may contain up to 99% by weight of the formula I compound. Solid carriers suitable for use in the composition of the invention include calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Any veterinary pharmaceutically acceptable liquid carrier suitable for preparing solutions, suspensions, emulsions, syrups and elixirs may be employed in the composition of the invention. The acetamide or carbonitrile compound may be dissolved or suspended in a veterinary pharmaceutically acceptable liquid carrier such as water, an organic solvent, or a veterinary pharmaceutically acceptable oil or fat, or a mixture thereof. Said liquid composition may contain other suitable veterinary pharmaceutical additives such as solubilizers, emulsifiers, buffers, preservatives, sweeteners, flavoring agents, suspending agents, thickening agents, coloring agents, viscosity regulators, stabilizers, osmo-regulators, or the like. Examples of liquid carriers suitable for oral and parenteral administration include water (particularly containing additives as above, e.g., cellulose derivatives, preferably sodium carboxymethyl cellulose solution), alcohols (including monohydric alcohols and polyhydric alcohols, e.g., glycols) or their derivatives, or oils (e.g., fractionated coconut oil and arachis oil). For parenteral administration the carrier may also be an oily ester such as ethyl oleate or isopropyl myristate.

Compositions of the invention which are sterile solutions or suspensions are suitable for intramuscular, intraperitoneal or subcutaneous injection. Sterile solutions may also be administered intravenously. Inventive compositions suitable for oral administration may be in either liquid or solid composition form.

For a more clear understanding of the invention, the following examples are set forth hereinbelow. These examples are merely illustrative and are not understood to limit the scope or underlying principles of the invention in any way. Indeed, various modifications of the invention, in addition to those shown and described herein, will become apparent to those skilled in the art from the examples set forth hereinbelow and the foregoing description. Such modifications are also intended to fall within the scope of the appended claims. Unless otherwise stated, all parts are parts by weight. The term mg/kg designates mg of test compound per kg of body weight of test animal. The term ml/kg designates ml of vehicle or test suspension per kg of body weight of test animal.

### EXAMPLE 1

### Evaluation of Plasma Concentration Profile of Test Compounds

In this evaluation 8 male Beagle breed dogs, 6 months to 2 years of age, and at least 10 kg in weight were used. Dogs were housed in individual indoor cages in compliance with standards outlined in the Guide for the Care and Use of Laboratory Animals of the Institute of Laboratory Animal resources, National Research Council. The dogs were fed an appropriate amount of standard commercial dry ration once per day. Fresh tap water in water bowls was provided ad libitum. On the day before the test compounds were administered, food was removed from the dogs by 4:00 pm. Food was withheld until after taking blood samples at the 4 h time point after administration of test materials. Dogs were weighed and ranked by descending weight order and were given physical exams by a veterinarian. The two heaviest dogs were randomly assigned to Group 1 or 2. The second two heaviest dogs were randomly assigned to Group 1 or 2 and so on, until all 8 dogs were placed into either of the two groups. Group 1 with 4 dogs/group, was treated orally with 15 mg/kg of the acetamide test compound and Group 2 was treated orally with 15 mg/kg of the carbonitrile test compound. One week after treatment all dogs were reweighed to ensure correct dosing. Two weeks after treatment, Group 1 was treated orally with 30 mg/kg of the acetamide test compound and Group 2 was treated orally with 30 mg/kg of the carbonitrile test compound. A treatment vial containing an appropriate amount of test compound was provided for each dog. The appropriate amount of vehicle (water containing 0.5% Methocel A4M and 0.01% polysorbate 80) was added to the vial and the contents of the vial were thoroughly mixed. The resultant suspension was withdrawn with an unarmed disposable 12 ml plastic syringe and administered to the dog. Test suspensions were administered to the back of the throat to ensure that they were reliably and completely swallowed by the dog. The vials were rinsed with 0.5 ml vehicle per kg of body weight and the rinsate was administered to the dog. Blood samples were collected at regular time intervals and analyzed for concentration of test compound. The Cₘₐₓ value (the maximum compound concentration in the plasma) and the Tₘₐₓ value (the time, after dosing, at which the peak compound concentration was obtained) were determined by observation. The calculated mean Tₘₐₓ and Cₘₐₓ values for each of the four treatments is shown in Table I below. For Table I, the term “Acetamide” designates N-methyl-N-[3-(3-methyl-1,2,4-triazolo-[4,3-b]pyridazin-6-yl)phenyl]acetamide; and the term “Carbonitrile” designates 7-(3-pyridyl)pyrazolo[1,5-a]pyrimidine-3-carbonitrile.

TABLE I

| | Dose | Tₘₐₓ | Cₘₐₓ |
|---|---|---|---|
| Treatment | mg/kg | hours | μg/ml |
| Acetamide | 15 | 0.50 | 5.59 |
| Acetamide | 30 | 0.88 | 12.22 |
| Carbonitrile | 15 | 4.25 | 3.70 |
| Carbonitrile | 30 | 8.25 | 6.39 |

### EXAMPLE 2

### Evaluation of Efficacy of Test Compounds

In this evaluation dogs were screened for noise phobic behaviors and 40 dogs were selected for testing. Male and female Beagles and Mongrels, 1-4.5 years of age and 6.4-18.4 kg body weight were used. Dogs were blocked by weight and by gender and randomly assigned to each of five treatment groups. Dogs were housed in individual indoor cages in a facility in compliance with standards outlined in the Guide for the Care and Use of Laboratory Animals of the Institute of Laboratory Animal Resources, National Research Council. The study was done in four phases, with each phase consisting of five treatment groups of two dogs each. Thus, each of the five treatment groups had eight replicates. Dogs were housed in individual pens and had ad libitum access to water. Food was removed from the dogs on the evenings prior to treatment (active and placebo). On the days when dogs were exposed to the noise stimulus, the dogs received food after the observation period ended. During the noise stimulus and observation periods there was minimal contact between the dogs and the observers. All personnel associated with the trial, with the exception of the Study Monitor, were blinded to the five treatments.

Screening:

Sixteen to twenty-six days prior to testing, a total of 144 dogs were screened for their response to a noise stimulus, which consisted of a 10 minute track of a CD, “Electrifying Thunderstorms”. All of the dogs were expected to startle when exposed to the thundersorm sounds on the CD. Those that exhibited particular behaviors and did not recover quickly from the startle were considered condidates for the study. Behaviors of interest included: panting, pacing, salivating, elimination (urination or defecation), withdrawal, trembling, running around frantically, vocalization, digging and scratching, freezing, and scanning. Efficacy of the test compounds was determined by the post-treatment reduction in the number and/or intensity of these behaviors upon exposure to the noise stimulus.

Definition of Behaviors:

Panting—breathing quickly or in a labored manner
Pacing—rhythmic, repetitive walking back and forth along the pen
Salivating—excess flow of saliva dripping off the dog's tongue, lips or mouth
Elimination—urination or defecation
Withdrawal—retreating or backing into a corner; dog is alert, non-sleeping
Trembling—shaking involuntarily (with fear)
Running around frantically—fast and nervous, disordered running or circling
Vocalization—barking, crying or whining
Digging and scratching—scraping or digging with the nails
Freezing—to become fixed or motionless
Scanning—back and forth or side to side head movement
   Test Procedure:

For treatment groups A-E in this procedure, the term “Acetamide” designates N-methyl-N-[3-(3-methyl-1,2,4-triazolo-[4,3-b]pyridazin-6-yl)phenyl]acetamide; and the term “Carbonitrile” designates 7-(3-pyridyl)pyrazolo[1,5-a]pyrimidine-3-carbonitrile.

Eleven to fifteen days prior to testing, Noise Phobia Screening Forms and videotapes of the dogs were reviewed and 40 dogs were selected for the study. Nine to ten days prior to testing, a veterinarian performed physical exams to ensure the dogs were healthy (e.g. showed no conditions that could produce the non-specific signs evaluated). Five days prior to testing, 40 dogs were weighed and blocked by weight and gender and randomly allocated to Treatment Groups A-E:
Group A: 15 mg/kg Acetamide
Group B: 5 mg/kg Acetamide
Group C: 15 mg/kg Carbonitrile
Group D: 5 mg/kg Carbonitrile
Group E: vehicle (placebo) water containing 0.5% Methocel A4M and 0.01% polysorbate 80

Phase 1 consisted of 10 dogs, 2 chosen randomly from each of the 5 treatment groups. For 3 days, ten dogs were acclimated to their environment (video monitored runs). On day 0, the 10 dogs were gavaged with 0.5 ml/kg vehicle. On day 1, the 10 dogs were gavaged with 0.5 ml/kg vehicle. On day 2, the 10 dogs were gavaged with 0.5 ml/kg vehicle and 1 h later were exposed to a 30 minute noise stimulus (“Electrifying Thunderstorms” CD). Dogs were closely monitored via video and observers for 3 h post gavage for the specific behaviors listed above, as well as side effects, including sedation, ataxia, vomiting, disorientation and diarrhea. The amount of time sleeping was also recorded since it was important to distinguish between sleeping (a relaxed state) and anxious behaviors, such as withdrawal. A rating system (0-3) was used for each behavior. Elimination was scored by counting the number of times an animal urinated or defecated during each 5 minute time period. On day 3, the 10 dogs were gavaged with 0.5 ml/kg vehicle. On day 4, the 10 dogs were gavaged with 0.5 ml/kg vehicle. On day 5, the dogs were gavaged with treatments, 2 dogs for each of treatments A-E. The test compounds were suspended in vehicle as described in Example1. The concentrations of the test compounds were prepared so that the volume given to each animal was 0.5 ml/kg. One h post treatment, the 30 minute noise stimulus was administered. Dogs were closely monitored via video and observers for 3 h post gavage for the specific behaviors listed above, as well as side effects, including sedation, ataxia, vomiting, disorientation and diarrhea. The amount of time sleeping was also recorded since it was important to distinguish between sleeping (a relaxed state) and anxious behaviors, such as withdrawal. A rating system (0-3) was used for each behavior. Elimination was scored by counting the number of times and animal urinated or defecated during each 5 minute time period. All observers were blinded with regard to treatments. Phase 2, Phase 3 and Phase 4, each consisting of 10 dogs not used in a previous phase, 2 from each of the 5 treatment groups, were run on subsequent weeks.

Results and Discussion:

Behaviors were analyzed by the one-sided Fisher's Exact test and by ANOVA procedures, comparing the differences in pretreated versus treated scores for each animal, as well as comparing the scores of all treatment groups to each other (only “after treatment”). Based on these analyses, there were significant treatment effects observed, particularly at the 15 mg/kg doses of each test compound. Behaviors demonstrating statistical significance included panting, trembling, withdrawal, scanning and vocalization. It is particulaly noteworthy that dogs treated with 15 mg/kg of test compound had significantly lower trembling scores, which the CD played, compared with the untreated control animals. Sedation, ataxia and disorientation were not observed for any of the treated dogs.

What is claimed is:

1. A method for the treatment and prevention of noise phobia in a companion animal which comprises providing said animal with a therapeutically effective amount of N-methyl-N-[3-(3-methyl-1,2,4-triazolo-[4,3-b]pyridazin-6-yl)phenyl]acetamide or 7-(3-pyridyl)pyrazolo[1,5-a]pyrimidine-3-carbonitrile.

2. The method according to claim 1 wherein the companion animal is selected from the group consisting essentially of a dog; cat; horse; hamster; and guinea pig.

3. The method according to claim 1 wherein the companion animal is a dog.

4. The method according to claim 3 which comprises providing said animal with a therapeutically effective amount of N-methyl-N-[3-(3-methyl-1,2,4-triazolo-[4,3-b]pyridazin-6-yl)phenyl]acetamide.

5. The method according to claim 1 wherein said effective amount is about 0.5 mg/kg-50 mg/kg.

6. The method according to claim 3 wherein said effective amount is about 5.0 mg/kg-40 mg/kg.

7. The method according to claim 3 wherein said acetamide or carbonitrile is provided orally.

8. A veterinary composition which comprises a vetereinary pharmaceutically acceptable carrier and an effective amount of N-methyl-N-[3-(3-methyl-1,2,4-triazolo-[4,3-b]pyridazin-6-yl)phenyl]acetamide or 7-(3-pyridyl)pyrazolo[1,5-a]pyrimidine-3-carbonitrile.

9. The compositon according to claim 8 which comprises an effective amount of N-methyl-N-[3-(3-methyl-1,2,4-triazolo-[4,3-b]pyridazin-6-yl)phenyl]acetamide.

10. The composition according to claim 8 wherein said veterinary pharmaceutically acceptable carrier is a liquid carrier.

11. The composition according to claim 8 wherein said veterinary pharmaceutically acceptable carrier is a solid carrier.

12. The composition according to claim 8 wherein the effective amount is sufficient to provide about 0.5 mg/kg-50 mg/kg of active ingredient.

13. The composition according to claim 9 wherein the effective amount is sufficient to provide about 5.0 mg/kg-40 mg/kg of active ingredient
